(19) United States
(12) Patent Application Publication
(10) Pub. No.: US 2026/0232250 A1
(43) Pub. Date: Aug. 13, 2026

(54) ELECTROANATOMICAL MAPPING SYSTEM USING AN OPTIMAL LEAD

(71) Applicant: Anumana, Inc., Cambridge, MA (US)

(72) Inventor: Animesh Agarwal, San Mateo, CA (US)

(73) Assignee: Anumana, Inc., Cambridge, MA (US)

(21) Appl. No.: 19/048,697

(22) Filed: Feb. 7, 2025

(51) Int. Cl. A61B 5/367; A61B 5/00; A61B 5/287; A61B 5/339

(52) U.S. Cl. CPC A61B 5/367; A61B 5/287; A61B 5/339; A61B 5/6823; A61B 5/6852; A61B 5/7246; A61B 5/7264; A61B 5/742; A61B 2560/0468

(57) ABSTRACT

An electroanatomical mapping system using an optimal lead. The system includes at least a processor and a memory communicatively connected to the at least a processor. The memory instructs the processor to receive a first potential signal from at least an electrode, wherein the first potential signal comprise a first voltage profile of a plurality of voltage profiles, select an optimal lead comprising the at least an electrode, identify a template beat, using the optimal lead, wherein the template beat comprises a first signal window of a plurality of signal windows of the first voltage profile, and automatically align, using the template beat and a beat matching algorithm, a second signal window of a second voltage profile of the potential signal to the first signal window of the first voltage profile to determine a first matched beat.

### FIELD OF THE INVENTION

The present invention generally relates to the field of electrophysiology. In particular, the present invention is directed to an electroanatomical mapping system using an optimal lead.

### BACKGROUND

During clinical electrophysiologic (EP) procedures on patients with cardiac arrhythmias, catheters with multiple electrodes are inserted, via veins or arteries, into cardiac chambers or tissues to record electrical signals (electrograms) that help the physicians diagnose and treat the patients. EP systems are used to record these signals, measure, and analyze signal metrics used to characterize the arrhythmias, assess the normality or abnormality of the heart tissue, and to plan a treatment strategy for the patient. Given that there can be many different and abnormal beats during these procedures, it is important to be able to detect and analyze data from only those beats specific to the arrhythmia. Existing solutions do not adequately solve this problem.

### SUMMARY OF THE DISCLOSURE

The method described in this document provides a powerful, robust, highly accurate means to ensure that a high percentage of only the specific beats needed are accepted for analysis. In an aspect, a system for determining a beat match using an optimal lead includes at least a processor and a memory communicatively connected to the at least a processor. The memory contains instructions configuring the processor to receive a first potential signal from the at least an electrode, wherein the first potential signal comprise a first voltage profile of a plurality of voltage profiles, select an optimal lead comprising the at least an electrode, identify a template beat, using the optimal lead, wherein the template beat comprises a first signal window of a plurality of signal windows of the first voltage profile, and automatically align, using the template beat and a beat matching algorithm, a second signal window of a second voltage profile of the potential signal to the first signal window of the first voltage profile to determine a first matched beat.

In another aspect, a method for an electroanatomical mapping system using an optimal lead includes receiving, using at least a processor, a first potential signal from at least an electrode, wherein the first potential signal comprise a first voltage profile of a plurality of voltage profiles, selecting, using the at least a processor, an optimal lead comprising the at least an electrode, identifying a template beat, using the optimal lead, wherein the template beat comprises a first signal window of a plurality of signal windows of the first voltage profile, and automatically aligning, using the template beat and a beat matching algorithm, a second signal window of a second voltage profile of the potential signal to the first signal window of the first voltage profile to determine a first matched beat.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is an illustration of a display of 6 electrocardiogram signals for several heart beats, a template window of the QRS for one beat, and a resulting detection signal;

FIG. 2 is an illustration of a histogram of the values of a detection signal that shows a most probable value;

FIG. 3 is an illustration of a detection signal for a first nine beats of a recording showing a most probable value, a 50% threshold, and a 75% threshold;

FIG. 4 is an illustration of an example of 100 consecutively detected beats of one ECG lead, stacked to show precise time alignment and waveform similarity;

FIG. 5 is an illustration of a flowchart showing a work flow for implementing a method of an apparatus;

FIG. 6 is a block diagram of an electroanatomical mapping system using an optimal lead;

FIG. 7A is an exemplary illustration of a first eigenvector distribution comprising optimal orthogonal leads, two lateral midlines, and an anterior midline;

FIG. 7B is an exemplary illustration of a second eigenvector distribution comprising optimal orthogonal leads, vectorcardiography leads, and 12-lead electrocardiogram leads;

FIG. 7C is an exemplary illustration of a second eigenvector distribution comprising optimal orthogonal leads, vectorcardiography leads, and 12-lead electrocardiogram leads;

FIG. 8A is an exemplary illustration of a comparison of graphs of optimal leads and vectorcardiography leads;

FIG. 8A is an exemplary illustration of a comparison of lead graphs of optimal leads and vectorcardiography leads;

FIG. 8B is an exemplary illustration of a comparison graphs of root mean square values of optimal leads and vectorcardiography leads;

FIG. 9 is a block diagram of an exemplary method for an electroanatomical mapping system using an optimal lead; and

FIG. 10 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

The system and method involves the recording and digitization of electrical signals generated by the heart from patients undergoing EP procedures. Continuing, the electrical signals may be recorded from a multiplicity (tens) of electrodes on and within the heart muscle and its chambers (electrograms or EGs) as well as from the patient's torso (electrocardiograms or EKGs). Without limitation, the electrical signals may be used to display a continuous record during the procedures and may be analyzed by computational methods to characterize the normality or abnormality of cardiac tissues, the specific, abnormal heart rhythm of the patient, and to provide the key information needed to treat the patient's specific abnormal rhythm. Without limitation, a problem that may arise in studying patients is that during procedures, many of the heart beats are not the same electrically, i.e., the electrical waves that propagate throughout the heart are not the same, beat-to-beat. Continuing, it is essential for the success of theses procedure that the electrical data from only very similar heart beats are used for the analyses. Without limitation, the method may describe ways to select those very similar beats.

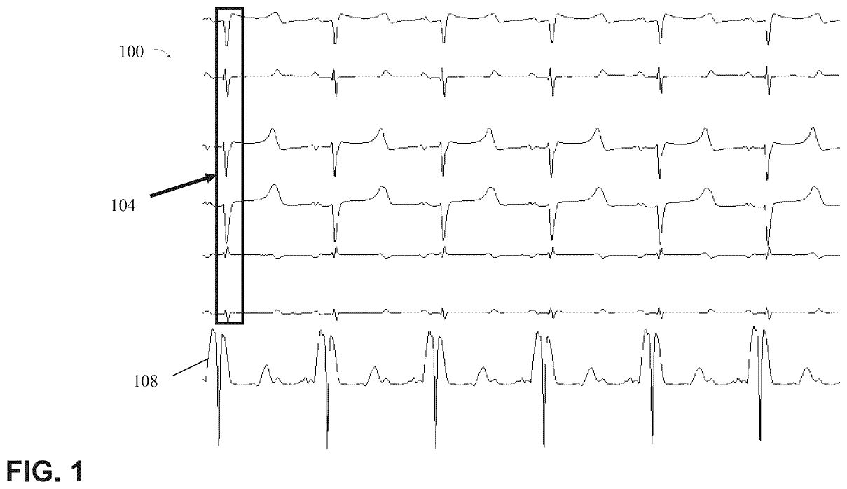

Referring now to FIG. 1, an illustration 100 of a display of 6 electrocardiogram signals for several heart beats, a template window of the QRS for one beat, and a resulting detection signal.

During an EP procedure, an EP system may be used to record and store many electrical signals from electrodes placed on and within the heart muscle and its cavities as well as from the body surface (torso). The P wave may reflect the electrical wave that propagates throughout the atrial heart tissue that triggers the contraction of the right and left atria. The QRS may reflect the electrical wave that propagates through the right and left ventricles.

With continued reference to FIG. 1, the first step of the method may include identifying, selecting, and windowing a P wave or QRS for a specific type of heart beat for the procedure. Without limitation, FIG. 1 illustrates 6 ECG leads and a windowing 104 of the QRS from one heartbeat. These are called the template P or template QRS against which all other beats during the procedure may be compared.

With continued reference to FIG. 1, the second step may include aligning the beginning of the template P or QRS with each sample of the signal window, one sample at a time across the entire recording being analyzed, and for each sample time, to generate a detection signal 108 that shows similarity of dissimilarity. Continuing, a simple, computationally efficient such signal may be used to calculate, for each time of alignment between the template and the signal stream, the sum of squared differences between the template signal and the data stream signal on a sample-by-sample basis across all EKG or EGM leads.

Let Ei(k) be the voltage of ECG or EGM I of N leads at sample time k. Let Ti(k) be the Template voltage of ECG or EGM I at time k, for k=1,NW where NW is number of samples of T. Let D(k) be the detection signal for time k where:

$$D ( k ) = {\sum }_{i = 1}^{NL} {\sum }_{n = 1}^{NW} {[ {E}_{i} ( k + n ) - {T}_{i} ( n ) ]}^{2} .$$

Without limitation, this may result in an error or detection signal 108, D(k), shown at the bottom of FIG. 1. Without limitation, the “Detection Signal” may show a very sharp, narrow downward deflection that identifies the time of closest template alignment with the data stream for every beat. Continuing, the minimum value of the signal, the nadir, provides the time of alignment for every beat and a metric of how exact the match is. Note that when the template is compared to the data stream containing the template, the detection signal 108 value may be zero. Note that the detection signal nadirs for non-template beats may have differing, non-zero values. Continuing, this may occur for two reasons: first, the propagating waves for each heartbeat of a given rhythm or arrhythmia may not be exactly the same, and second, during the respiratory cycles of the patient, the torso's electrical conductivity may change resulting in changes of ECG and EGM voltages.

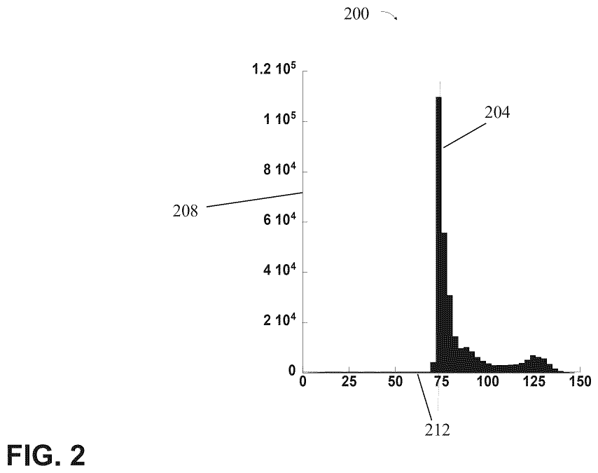

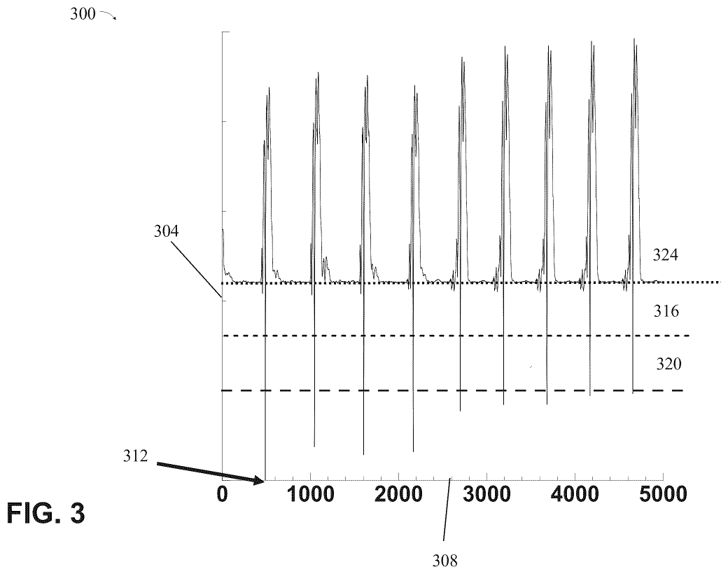

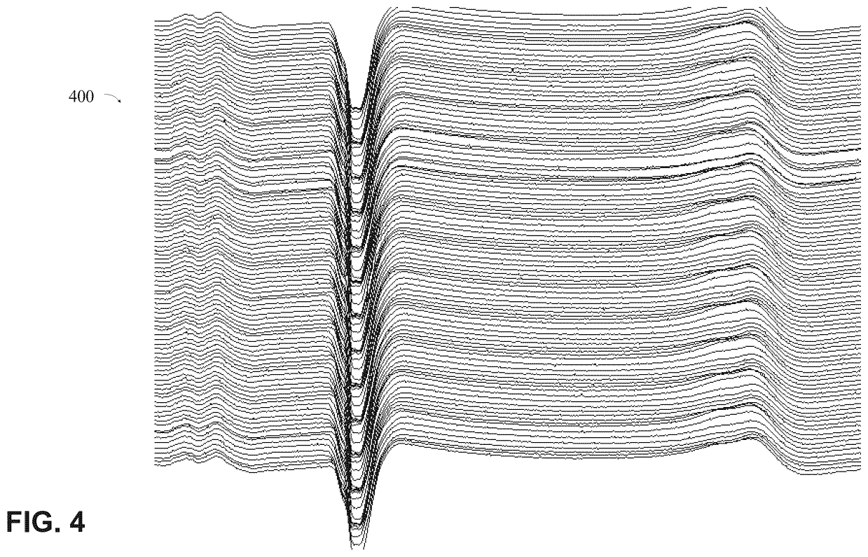

With continued reference to FIG. 1, without limitation, the third step may be to gather for several beats (a few tens), statistics of the detection signal 108 samples can be calculated and compared to for example, the most probable or median values of the entire detection signal—something close to the average or baseline of the signal. FIG. 2 illustrates a histogram of the detection signal samples and shows the most probable value of the detection signal as described in more detail below. FIG. 3 shows the detection signal, the most probable value (baseline) and the 50% and 75% values that can be used as threshold as described in more detail below. Continuing, this may allow the physician performing the procedure to select near identical or more loosely identical beats depending on the case. Without limitation, once a beat has been detected, and accepted, all the data recorded from all the electrodes used in the study can be used to analyze the tissue and the characteristics of the specific rhythm being studied. FIG. 4 illustrates one ECG lead with 100 consecutively selected beats stacked to show alignment and similarity of beats as described in more detail below.

Referring now to FIG. 2, an illustration 200 of a histogram of the values of a detection signal that shows a most probable value. In an embodiment, the illustration 200 may include a histogram of detection signal 204. In an embodiment, the histogram of detection signal 204 may include a “Count-Number of Samples” 208 as the X-axis. In an embodiment, the histogram of detection signal 204 may include a “Detection Signal Amplitude” as the Y-axis 212.

Referring now to FIG. 3, an illustration 300 of a detection signal for a first nine beats of a recording showing a most probable value, a 50% threshold, and a 75% threshold. In an embodiment, the illustration 300 may include a “Detection Signal Amplitude” 304 as the X-axis. In an embodiment, the illustration 300 may include a “Time” 308 in milliseconds as the Y-axis. In an embodiment, the illustration 300 may include a “Template Nadir” 312. In an embodiment, the illustration 300 may include a 75% threshold 316. In an embodiment, the illustration 300 may include a 50% threshold 320. In an embodiment, the illustration 300 may include a most probable value 324.

Referring now to FIG. 4, an illustration 400 of an example of 100 consecutively detected beats of one ECG lead, stacked to show precise time alignment and waveform similarity. In an embodiment, the illustration 400 may include 100 consecutively detected beats of one ECG Lead (time aligned to detection signal minima).

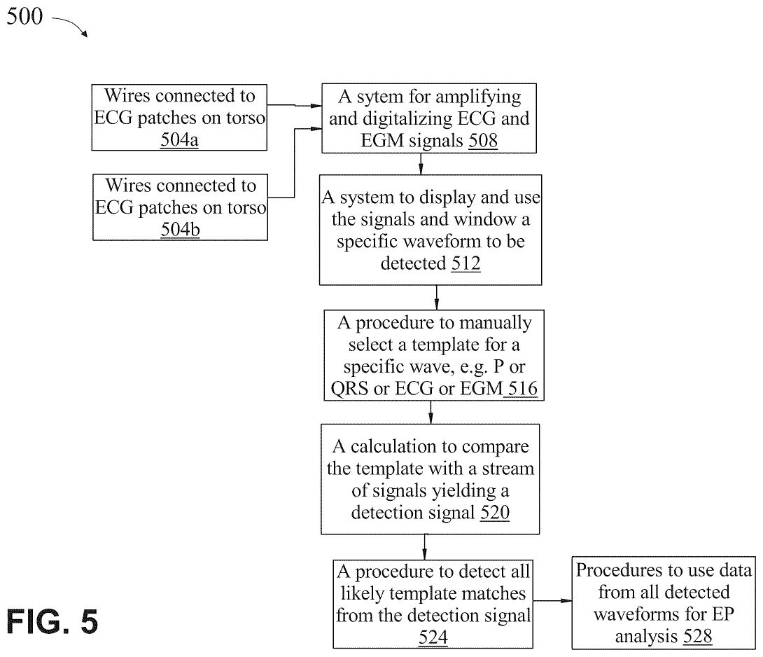

Referring now to FIG. 5, an illustration 500 of a flowchart showing a work flow for implementing a method of an apparatus. In an embodiment, the method uses one or more ECGs from the patient's torso 504a-b for the calculations. In an embodiment, the method uses a system for amplifying and digitalizing ECG and EGM signals 508. In an embodiment, the method uses a system to display and use the signals and window a specific waveform to be detected 512. In an embodiment, the method uses a procedure to manually select a template for a specific wave 516. For example the template for a specific wave 516 may include the P wave or the QRS wave, ECG, or EGM. In an embodiment, the method uses a calculation to compare the template with a stream of signals yielding a detection signal 520. In an embodiment, the method uses a procedure to detect all likely template matches from the detection signal 524. In an embodiment, the method uses procedures to use data from all detected waveforms for EP analysis 528.

In an embodiment, the method uses EGMs from electrodes on one or more catheters placed in the heart or in the coronary sinus, the cardiac vein between the atria and ventricles. In an embodiment, the method uses the ECG and or EGM electrical signals for the template and signal window to be analyzed. In an embodiment, the method uses the first derivatives of the ECGs and or EGMs for the template and signal waveforms. Continuing, this embodiment may include improve accuracy of detecting the correct beats. In an embodiment, the method uses smoothed or filtered versions of all the signals to reduce noise and improve signal-to-noise ratio that improves accuracy and reliability of correct beat detection. In an embodiment, the more EKGs or EGs used, the more robust the detection signal is detecting and accepting beats for study. In an embodiment, the method is to select a small number of optimally selected ECG or EGM signals that reduces the number of leads needed for accurate template matching. Selecting leads that have the highest signal magnitude and that on average have the least correlation to other selected leads, this improves computational efficiency and minimizes redundancy in the signals.

At a high level, aspects of the present disclosure are directed to an electroanatomical mapping system using an optimal lead. The system includes at least a computing device comprised of a processor and a memory communicatively connected to the processor. The memory instructs the processor to receive a first potential signal from at least an electrode, wherein the first potential signal comprise a first voltage profile of a plurality of voltage profiles. The processor selects an optimal lead comprising the at least an electrode. The processor identifies a template beat, using the optimal lead, wherein the template beat comprises a first signal window of a plurality of signal windows of the first voltage profile. Additionally, the processor automatically aligns, using the template beat and a beat matching algorithm, a second signal window of a second voltage profile of the potential signal to the first signal window of the first voltage profile to determine a first matched beat.

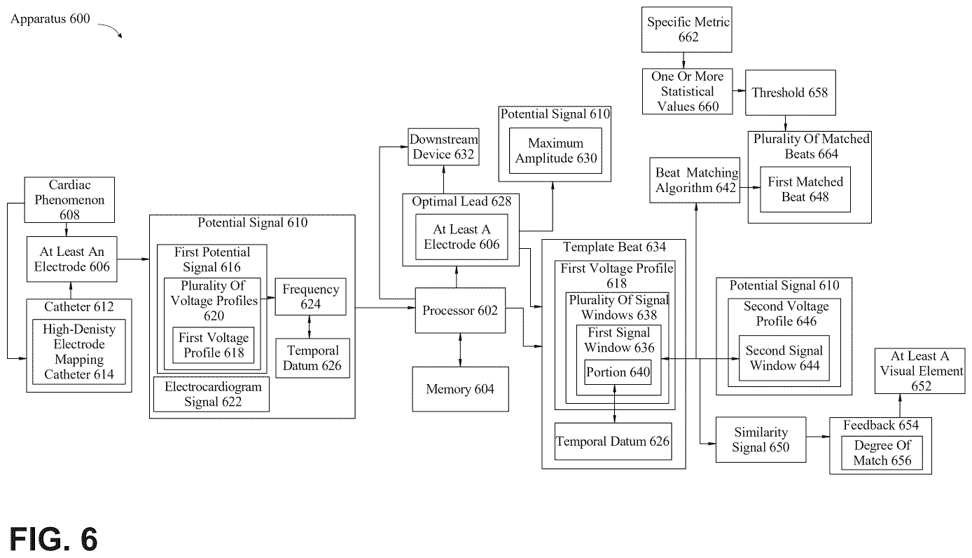

Referring now to FIG. 6, an exemplary embodiment of the electroanatomical mapping system 600 using an optimal lead is illustrated. System 600 may include a processor 602 communicatively connected to a memory 604. As used in this disclosure, “communicatively connected” means connected by way of a connection, attachment, or linkage between two or more relata which allows for reception and/or transmittance of information therebetween. For example, and without limitation, this connection may be wired or wireless, direct or indirect, and between two or more components, circuits, devices, systems, and the like, which allows for reception and/or transmittance of data and/or signal(s) therebetween. Data and/or signals there between may include, without limitation, electrical, electromagnetic, magnetic, video, audio, radio and microwave data and/or signals, combinations thereof, and the like, among others. A communicative connection may be achieved, for example and without limitation, through wired or wireless electronic, digital or analog, communication, either directly or by way of one or more intervening devices or components. Further, communication connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

With continued reference to FIG. 6, memory 604 may include a primary memory and a secondary memory. “Primary memory” also known as “random access memory” (RAM) for the purposes of this disclosure is a short-term storage device in which information is processed. In one or more embodiments, during use of the computing device, instructions and/or information may be transmitted to primary memory wherein information may be processed. In one or more embodiments, information may only be populated within primary memory while a particular software is running. In one or more embodiments, information within primary memory is wiped and/or removed after the computing device has been turned off and/or use of a software has been terminated. In one or more embodiments, primary memory may be referred to as “Volatile memory” wherein the volatile memory only holds information while data is being used and/or processed. In one or more embodiments, volatile memory may lose information after a loss of power. “Secondary memory” also known as “storage,” “hard disk drive” and the like for the purposes of this disclosure is a long-term storage device in which an operating system and other information is stored. In one or remote embodiments, information may be retrieved from secondary memory and transmitted to primary memory during use. In one or more embodiments, secondary memory may be referred to as non-volatile memory wherein information is preserved even during a loss of power. In one or more embodiments, data within secondary memory cannot be accessed by processor. In one or more embodiments, data is transferred from secondary to primary memory wherein processor 602 may access the information from primary memory.

Still referring to FIG. 6, system 600 may include a database. The database may include a remote database. The database may be implemented, without limitation, as a relational database, a key-value retrieval database such as a NOSQL database, or any other format or structure for use as database that a person skilled in the art would recognize as suitable upon review of the entirety of this disclosure. The database may alternatively or additionally be implemented using a distributed data storage protocol and/or data structure, such as a distributed hash table or the like. The database may include a plurality of data entries and/or records as described above. Data entries in database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in database may store, retrieve, organize, and/or reflect data and/or records.

With continued reference to FIG. 6, system 600 may include and/or be communicatively connected to a server, such as but not limited to, a remote server, a cloud server, a network server and the like. In one or more embodiments, the computing device may be configured to transmit one or more processes to be executed by server. In one or more embodiments, server may contain additional and/or increased processor power wherein one or more processes as described below may be performed by server. For example, and without limitation, one or more processes associated with machine learning may be performed by network server, wherein data is transmitted to server, processed and transmitted back to computing device. In one or more embodiments, server may be configured to perform one or more processes as described below to allow for increased computational power and/or decreased power usage by the system computing device. In one or more embodiments, computing device may transmit processes to server wherein computing device may conserve power or energy.

Further referring to FIG. 6, system 600 may include any “computing device” as described in this disclosure, including without limitation a microcontroller, microprocessor, digital signal processor (DSP) and/or system on a chip (SoC) as described in this disclosure. System 600 may include, be included in, and/or communicate with a mobile device such as a mobile telephone or smartphone. System 600 may include a single computing device operating independently, or may include two or more computing devices operating in concert, in parallel, sequentially or the like; two or more computing devices may be included together in a single computing device or in two or more computing devices. System 600 may interface or communicate with one or more additional devices as described below in further detail via a network interface device. Network interface device may be utilized for connecting processor 602 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Processor 602 may include but is not limited to, for example, a computing device or cluster of computing devices in a first location and a second computing device or cluster of computing devices in a second location. System 600 may include one or more computing devices dedicated to data storage, security, distribution of traffic for load balancing, and the like. System 600 may distribute one or more computing tasks as described below across a plurality of computing devices of computing device, which may operate in parallel, in series, redundantly, or in any other manner used for distribution of tasks or memory between computing devices. System 600 may be implemented, as a non-limiting example, using a “shared nothing” architecture.

With continued reference to FIG. 6, processor 602 may be designed and/or configured to perform any method, method step, or sequence of method steps in any embodiment described in this disclosure, in any order and with any degree of repetition. For instance, processor 602 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Processor 602 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 6, the system 600 includes at least a surface electrode pair 606 configured to detect at least a surface potential signal 610, comprising a surface electrocardiogram (ECG), as a function of a cardiac phenomenon 608 of a subject and be orthogonally placed on a torso of the subject in locations that maximize an average signal to noise ratio of the at least a surface potential signal 610. As used in this disclosure, the “torso” is the central part of the human body, excluding the head, neck, and limbs. Without limitation, the torso may include the anterior (front) and posterior (back) portions of the body. As used in this disclosure, an “electrode” is a conductor through which electrical signals enter or exit a medium. Without limitation, the at least a surface electrode pair 606 may detect, measure, or transmit electrical activity from the body, such as the heart or brain. Electrodes and transducers may be used together in medical devices where the electrodes detect and record electrical signals from the body (e.g., heart activity), while transducers convert other forms of energy (e.g., ultrasound or pressure) into electrical signals for imaging or measuring physical parameters, enabling simultaneous monitoring of electrical and mechanical functions. As used in this disclosure, a “transducer” is a device designed to convert one form of energy into another. In a non-limiting example, transducer may facilitate the measurement, monitoring, and control of various physical quantities. Without limitation, this energy conversion capability may enable transducers to be used for various applications. In a non-limiting embodiment, a transducer may detect at least a cardiac phenomenon 608 and output at least a surface potential signal 610. As used in this disclosure, a “cardiac phenomenon” is any physiological or pathological event, activity, or condition related to the function or behavior of the heart that can be detected or measured. The cardiac phenomenon 608 includes but is not limited to electrical signals, mechanical movements, pressure changes, and/or biochemical processes occurring within the heart or its surrounding tissues. The cardiac phenomenon 608 may be crucial indicators of heart health and function and may provide valuable data for diagnosing, monitoring, and treating various cardiac conditions. In a non-limiting example, a cardiac phenomenon 608 may refer to the electrical activity associated with the heart's rhythm, such as the depolarization and repolarization of cardiac cells that create the P wave, QRS complex, and T wave observed in an electrocardiogram (ECG or EKG). In another non-limiting example, the transducer may include a plurality of clinical transducers. As used in this disclosure, a “plurality of clinical transducers” is a transducer device used in the medical field to measure, analyze, and/or quantify electrical signals in a body. As used in this disclosure, “potential signal” is electrical signals generated and output by a transducer in response to detecting cardiac phenomenon 608. Without limitation, the at least a surface potential signal 610 may be indicative of the heart's electrical activity. Without limitation, the at least a surface potential signal 610 may represent variations in electrical potential that occur as the heart undergoes its rhythmic contractions and relaxations, providing valuable data on the cardiac cycle and function.

With continued reference to FIG. 6, the at least a transducer may be coupled to a lead. A “lead,” as used in this disclosure, is one or more electrodes attached to the skin to detect a heart's electric signals. Without limitation, the system 600 may include a standard 12-lead configuration. As used in this disclosure, a “standard 12-lead configuration” is a measurement the electrical activity of a heart from 12 different perspectives. In a non-limiting embodiment a standard 12-lead electrocardiogram signal 622 may include a graphical record of the direction and magnitude of the electrical activity generated by the depolarization and repolarization of the atria and ventricles of the heart. Without limitation, the system 600 may include various lead configurations.

With continued reference to FIG. 6, the system 600 may include different hardware for specific measurements. In some embodiments, hardware may be transducers, sensors, and actuators. For the purposes of this disclosure, a “sensor” is a device used to transform one kind of energy into another. When a transducer converts a quantity of energy to an electrical voltage or an electrical current it is called a sensor. A measurable quantity of energy may include sound pressure, optical intensity, magnetic field intensity, thermal pressure, etc. When a transducer converts an electrical signal into another form of energy such as sound, light, mechanical movement, it is called an actuator. It should be noted that sound is incidentally a pressure field. Actuators allow the use of feedback at the source of the measurements.

With continued reference to FIG. 6, a sensor may be considered as a component or with a collection of electronics such as amplifiers, decoders, filters, computer devices and the system 600. For the purposes of this disclosure an “instrument” is a sensor bundled with its associated electronics. However, in some embodiments, sensors may be further integrated with the system 600.

With continued reference to FIG. 6, a sensor integrated with the system 600 may be linear so that response y to a stimulus x is in the form: y(x)=Ax, 0≤x≤xₘₐₓ, A>0. It should be noted, there is a presumption that the stimulus to be positive. A is the sensitivity of the transducer gain, or the gain of the sensor. The gain is presumed to be positive for which the linear model satisfies the definition of linearity: y(x+z)=A(x+z)=y(x)+y(z). It should be noted that this example is an idealized form of a sensor and may extend beyond the linearity constraints which may include time dependency, memory, and its output keeping track of input. A more generalized sensor may include the steady state transfer function of the sensor. For this case, the sensitivity can be defined as the derivative of the output with respect to the input:

$$S = \frac{\partial y}{\partial x} .$$

In this example, the sensor exhibits sensitivities to other operating parameters (i.e. supply voltage) or temperature. For the purposes of this disclosure, “sensitivity” is the ratio of output to input. This can include electrical output and signal input or an input transducer. It can also include physical output to an electrical input, or an output transducer. Sensitivity can also be used in its usual electrical meaning. In this it would refer to a percent change of a property of a device because of a percent change in a parameter. In some embodiments this would be a percent change in gain as a result of percent change in ambient temperature. This type of sensitivity may be referred to as the Gain of a sensor.

Still referring to FIG. 6, the system 600 with integrated sensors may not respond to arbitrarily small signals. The system 600 may respond to signals within a specified range from zero to a sensor threshold which does not cause the output of the sensor to change. The existence of a threshold relates to the nonlinear behavior of the device and the noise. The system 600 with an integrated sensor may fail to respond to stimuli which are arbitrarily large as well. In this case, the system 600 integrated with a sensor may have a max range. The full range of the system 600 integrated with a sensor may be limited by compression or clipping. Compression and clipping are results of nonlinearity and thus may include the system 600 as a nonlinearity device.

Still referring to FIG. 6, referring to the linear equation above assuming a linear sensor is improved with the addition of a constant: y(x)=b₀+Ax. It should be noted that the equation is not linear even though it is described as a first order polynomial. The constant is called a zero offset and can be defined in two ways: a sensor reading when the input is zero, or the value of the stimulus required to make the output zero. The zero offset is corrected by subtracting b₀ from y and recovering the linear description of a sensor: y′(x)=y(x)−b₀=Ax.

With continued reference to FIG. 6, the system 600 may include very fast measurements where it can internally store energy. The system 600 output may depend on previous measurements the integrated sensors make. It should be noted that the sensor may exhibit memory. The time dependence of a sensor can be linear if the response is described by a linear differential equation:

$${\sum }_{n = 0}^{N} {A}_{n} \frac{{\partial }^{n} y}{\partial {t}^{n}} = {\sum }_{k = 0}^{k} {B}_{k} \frac{{\partial }^{k} x}{\partial {t}^{k}} .$$

Taking the Laplace transform of this equation:

$$y ( s , X ) = ( \frac{{\sum }_{k = 0}^{K} {B}_{k} {S}^{k}}{{\sum }_{n = 0}^{N} {A}_{n} {S}^{n}} ) x = H ( s ) X ( s ) ,$$

which is in Laplace transform space and the sensor response is still linear in stimulus x. The response of a sensor with a transfer function H(s) at time t is the convolution integral between the history of the stimulus x and the inverse Laplace transform

$$h ( t ) of H ( s ) : y ( t ) = {\int }_{0}^{∞} h ( τ ) x ( t - τ ) d τ .$$

The system 600 may behave like a low pass filter, wherein there is a delayed response to their input. There is a limit to the maximum stimulus frequency that can be detected. The maximum frequency a sensor can interpret is approximately the inverse of its response time.

With continued reference to FIG. 6, the surface electrode pair may be disposed on the torso located at a first intersection on an anterior midline and slightly above a transversal plane and at a second intersection substantially between the anterior midline and a right lateral midline and slightly below the transversal plane. As used in this disclosure, an “intersection” is a location or point at which two or more defined elements meet or cross for the first time. In a non-limiting example, the elements may include lines, paths, geometric features, planes, axis, and the like. As used in this disclosure, the “transversal plane” is an imaginary horizontal plane that spans across the middle of the torso. In an embodiment, the transversal plane may be perpendicular to the vertical lines such as the anterior midline, posterior midline, and right lateral midline. As used in this disclosure, an “anterior midline” is an imaginary line running vertically down the front of a structure or object. As used in this disclosure, “substantially” indicates a position, value, or relationship that is nearly achieved. Without limitation, substantially may indicate a minor deviation or variation that does not materially affect the intended function or result, such as 0-0.5 inches away from the referenced position. As used in this disclosure, “slightly” indicates a small degree, extent, or deviation from a specified position, value, or relationship. Without limitation, slightly may indicate a deviation from the referenced position of 0.5-3 inches. In a non-limiting example, substantially may refer to a position within approximately 0.3 inch of the transversal plane of an anterior midline, allowing for minor variations while maintaining alignment with the intended anatomical reference point. In another non-limiting example, slightly may refer to a position approximately 2 inches below the transversal plane, indicating a small but noticeable deviation from the specified reference point. As used in this disclosure, “moderately” is a degree or extent that is larger than slightly as described herein. For example, without limitation, moderately may include 3 to 5 inches away from a given plane, axis, reference element, and the like. In an embodiment, the surface electrode pair may be disposed on the torso located at a third intersection slightly right of the anterior midline and substantially on the transversal plane and at a fourth intersection on a posterior midline and on the transversal plane. In an embodiment, the surface electrode pair may be disposed on the torso located at a fifth intersection slightly left of the anterior midline and moderately below the transversal plane and a sixth intersection moderately right of the anterior midline and moderately above the transversal plane.

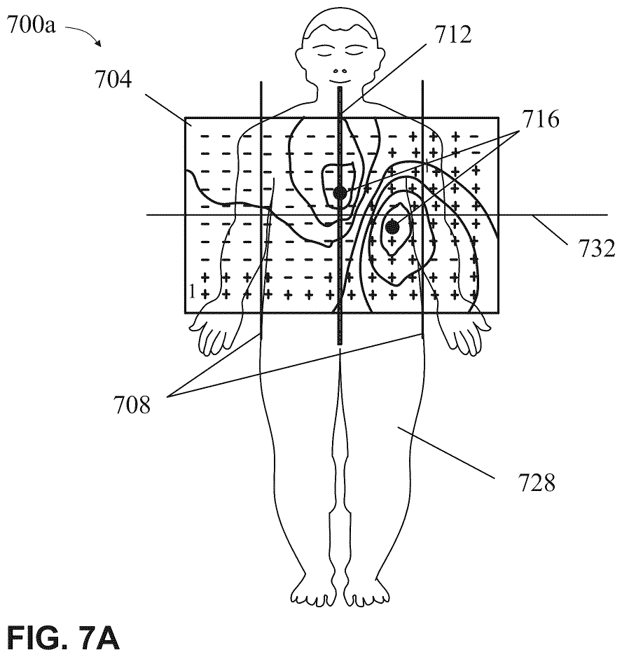

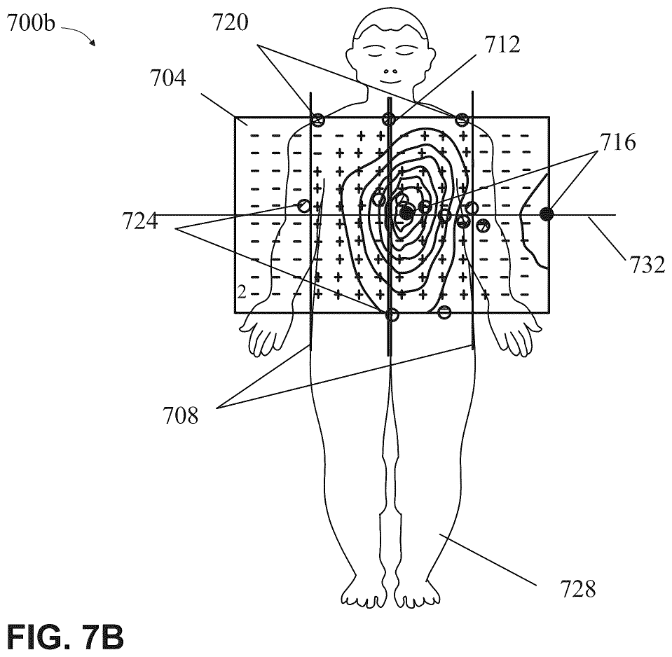

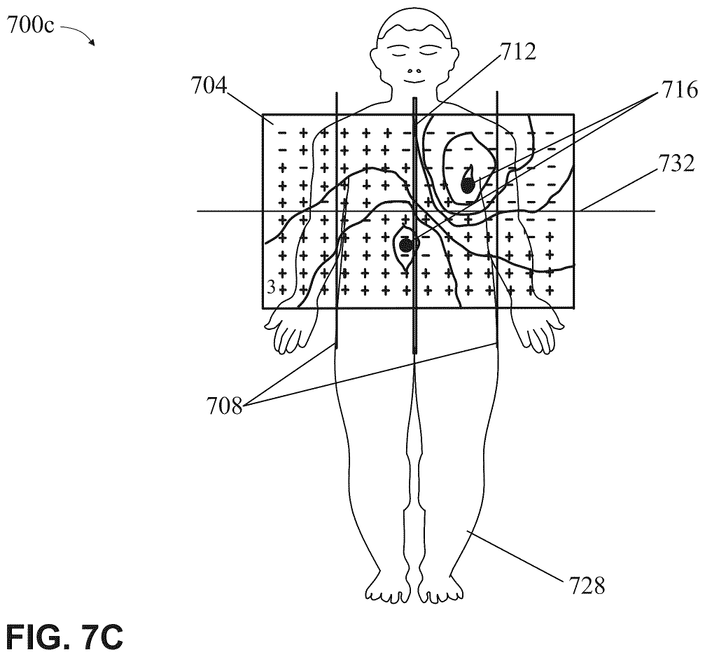

With continued reference to FIG. 6, the surface electrode pair may be disposed on the torso substantially located at a transversal axis of an anterior midline and slightly below the transversal axis between the anterior midline and a right lateral midline. As used in this disclosure, a “posterior midline” is an anatomical reference line located along the back (posterior) surface of the body, extending longitudinally along the central axis. Without limitation, the posterior midline may serve as a point of symmetry or reference for anatomical structures. In an embodiment, the surface electrode pair may be disposed on the torso substantially located at a position slightly below the transversal axis of the anterior midline and at a top slightly left justified location between the anterior midline and a right lateral midline. Refer to FIGS. 7A-C for an exemplary illustration of the surface electrode pair locations on a torso.

Still referring to FIG. 6, the system 600 includes a catheter 612 configured for intracardiac use and comprising at least a cardiac electrode pair configured to detect at least a cardiac potential signal, comprising a cardiac electrogram (EGM), as a function of the cardiac phenomenon. As used in this disclosure, a “catheter” is a tube inserted into the body to perform various medical procedures. In a non-limiting example, at least a catheter 612 may record and map at least a beat of a cardiac phenomenon 608 and output at least a visual element 652. In a non-limiting example, at least a catheter 612 may be used to facilitate the detection and mapping of cardiac activity. In a non-limiting example, at least a catheter 612 may be used in procedures such as cardiac ablation or electrophysiological studies to gather detailed information about heart rhythms. Without limitation, the catheter 612 may include one or more electrodes. As used in this disclosure, a “cardiac electrode pair” is a set of two electrodes positioned in proximity to cardiac tissue to detect, measure, or deliver electrical signals. Without limitation, one electrode of the cardiac electrode pair may serve as the reference, and the other electrode of the cardiac electrode pair may serve as the active electrode, allowing the measurement of electrical potential differences associated with cardiac activity. As used in this disclosure, a “cardiac potential signal” is an electrical signal generated by the heart due to the depolarization and repolarization of cardiac muscle cells. Without limitation, the cardiac potential signal may reflect the heart's electrical activity and may be fundamental to the initiation and coordination of myocardial contraction, which enables effective blood circulation. As used in this disclosure, a “cardiac electrogram” is an electrical recording of cardiac potential signals captured directly from electrodes placed on or within the heart.

Without limitation, the at least a surface electrode pair 606 configuration may produce an electroanatomical map of the heart. As used in this disclosure, an “electroanatomic map” is a detailed, three-dimensional representation of the electrical activity and anatomical structure of the heart. In a non-limiting example, the electroanatomic map may be created using data collected from a catheter 612 that records and maps cardiac phenomena. In another non-limiting example, the electroanatomic map may provide a visual depiction of the heart's electrical impulses and physical form, enabling precise identification and analysis of areas that may be causing abnormal heart rhythms or other cardiac issues. Continuing, the electroanatomic map may integrate both the electrical signals and the spatial geometry of the heart, offering a comprehensive tool for diagnosis and treatment planning. In a non-limiting example, an electroanatomic map may be created during an electrophysiological study where a catheter 612 is navigated through the heart to record electrical activity. The data collected from various points within the heart is used to construct a three-dimensional map that highlights regions of interest, such as areas with abnormal electrical pathways or scar tissue. This map can be displayed on a monitor, providing clinicians with a visual guide to target specific areas for ablation therapy, thereby improving the precision and effectiveness of the treatment. In another non-limiting example, the electroanatomic map may be employed during a cardiac procedure to continuously update the map in real-time as at least a catheter 612 moves within the heart. This dynamic mapping allows for immediate adjustments based on the current electrical activity and anatomical changes observed during the procedure. Such real-time updates may be particularly useful in complex cases where the anatomy and electrical activity of the heart vary significantly from patient to patient, ensuring that the intervention is tailored to the individual's specific cardiac structure and function.

With continued reference to FIG. 6, the at least a surface potential signal 610 may include electrograms (EGMs). As used in this disclosure, “electrograms” are the electrical recordings of cardiac activity captured from electrodes. In a non-limiting example, the electrodes used to capture the electrograms may be placed either on the surface of the heart, within the heart, or in proximity to the heart. Continuing, the electrograms may represent the electrical signals generated by the depolarization and repolarization of heart muscle cells during each heartbeat. In a non-limiting example, electrograms may be used to analyze heart rhythms, diagnose arrhythmias, and guide procedures such as catheter 612 ablation, electrophysiological studies, and the like. Without limitation, the electrograms may be collected using the catheter 612.

With continued reference to FIG. 6, the catheter 612 may include a high-density electrode mapping catheter 614. As used in this disclosure, a “high-density electrode mapping catheter” is a medical device designed to include a number of closely spaced electrodes along its surface. In a non-limiting example, the high-density electrode mapping catheter 614 may be used for creating detailed electrical maps of cardiac tissue. Continuing, the high-density electrode mapping catheter 614 may be inserted into the heart during an electrophysiological procedure to collect high-resolution data about the electrical activity of the heart's chambers. Continuing, the dense arrangement of electrodes on the high-density electrode mapping catheter 614 may allow for more precise and detailed recordings.

With continued reference to FIG. 6, the high-density electrode mapping catheter 614 may include a basket catheter, a grid catheter, a linear catheter, a loop catheter and the like. Continuing, the high-density electrode mapping catheters 614 may be designed for specific purposes in cardiac electrophysiology to enhance the precision of mapping and treatment of arrhythmias. These catheters vary in design, flexibility, electrode count, and configuration to suit different parts of the heart and various medical needs. As used in this disclosure, a “basket catheter” is a high-density electrode mapping catheter 614 with a flexible, basket-like structure composed of multiple splines (arms). In a non-limiting example, the splines of the basket catheter may expand when inserted into a heart chamber. Continuing, the basket catheter may include electrodes that are distributed along the splines to capture electrical signals from a wide area of the chamber. Continuing, the structure of the basket catheter may permit the catheter to conform to the shape of the chamber, providing detailed three-dimensional mapping of electrical activity across large regions of the heart. As used in this disclosure, a “grid catheter” is a catheter featuring a flat or grid-like arrangement of electrodes. Continuing, the grid catheter may include electrodes that are closely spaced, providing high spatial resolution for capturing detailed electrical data. Continuing, the structure of the grid catheter may provide particularly useful in detecting conduction abnormalities, such as areas of scar tissue or regions of abnormal electrical activity. As used in this disclosure, a “linear catheter” is a type of mapping catheter with electrodes arranged in a straight line along the catheter's shaft. In a non-limiting example, the linear catheter may be designed for mapping specific, narrow regions of the heart, such as along the septum or the pathways near veins and arteries. Continuing, the design of the linear catheter may allow for detailed analysis of conduction paths in confined areas. As used in this disclosure, a “loop catheter” is a catheter that forms a loop with multiple rings or arcs of electrodes around its structure. Without limitation, the loop catheter may expand once positioned in the heart, allowing for circumferential contact with the chamber walls, thus providing extensive coverage of the electrical signals within the chamber.

Still referring to FIG. 6, processor 602 is configured to receive a first potential signal 616 from the at least a surface electrode pair 606, wherein the first potential signal 616 comprise a first voltage profile 618 of a plurality of voltage profiles 620. As used in this disclosure, a “voltage profile” is a representation of the variation in voltage over time or across a particular component, illustrating how the voltage fluctuates under different operational conditions or as part of a specific electrical signal. The voltage profile may include characteristics such as amplitude, frequency, and waveform shape, which are indicative of the behavior of the system being monitored. In a non-limiting example, the voltage profile may describe how the voltage generated by the heart's electrical activity changes over time. For instance, without limitation, during a single heartbeat, an electrocardiogram may capture the voltage fluctuations that occur as electrical impulses travel through the heart muscle. Continuing, this voltage profile may include distinct phases, such as P wave, QRS complex, and T wave. Without limitation, the P wave may include a small increase in voltage representing atrial depolarization. Without limitation, the QRS complex may include a sharp rise and fall in voltage reflecting the depolarization of the ventricles. Continuing, the T wave may include a slower increase and decrease in voltage due to ventricular repolarization. Without limitation, the voltage profile of an ECG waveform may vary over time, showing the precise moments of electrical activity in the heart.

Still referring to FIG. 1, the at least a processor 602 is configured to receive the at least a surface potential signal from the at least a surface electrode pair. In a non-limiting example, the at least a processor 602 may be designed to interact with the pair of surface electrodes. These surface electrodes generate a signal representing surface potential, which is subsequently received and processed by the processor 602.

Still referring to FIG. 1, the at least a processor 602 is configured to receive the at least a cardiac potential signal from the at least a cardiac electrode pair and synchronize the at least a cardiac potential signal from the at least a cardiac electrode pair with the at least a surface potential signal. As used in this disclosure, “synchronize” is the process of aligning or coordinating multiple signals, data streams, or events in time or sequence to ensure consistent temporal or functional correlation. Without limitation, the alignment of the at least a cardiac potential signal from the at least a cardiac electrode pair with the at least a surface potential signal to facilitate accurate analysis, comparison, or further processing of these signals.

With continued reference to FIG. 6, in a non-limiting example, the system 600 may include multiple electrodes placed on or inside a human body or a heart. Continuing, each of the at least a surface electrode pair 606, may detect electrical signals or voltage changes. Without limitation, the first voltage profile 618 may represent the specific electrical activity captured by one of these electrodes at a given moment. Continuing, the processor may collect the first voltage profile 618 as part of a larger data set. Without limitation, the first voltage profile 618 may include information about the amplitude and timing of electrical signals. In a non-limiting example, the first voltage profile 618 may provide insight into the physiological or electrical characteristics of the area being monitored. Without limitation, the plurality of voltage profiles 620 demonstrates that the processor may receive the voltage profiles from multiple electrodes, where each of the at least a surface electrode pair 606 may detect and transit its own voltage profile to the processor for comprehensive analysis of the entire area being examined.

With continued reference to FIG. 6, the at least a surface potential signal 610 may include an electrocardiogram signal 622. As used in the current disclosure, an “electrocardiogram” is a signal representative of electrical activity of heart over time. Without limitation, the electrocardiogram signal 622 may capture voltage changes in the voltage profile, reflecting the heart's electrical impulses as they propagate through cardiac tissue. Without limitation, the ECG may be used to analyze the heart's rhythm and detect abnormalities in cardiac function. Without limitation, electrocardiograms may consist of several distinct waves and intervals, each representing a different phase of the cardiac cycle. These waves may include the P-wave, QRS complex, T wave, U wave, and the like. The P-wave may represent atrial depolarization (contraction) as the electrical impulse spreads through the atria. The QRS complex may represent ventricular depolarization (contraction) as the electrical impulse spreads through the ventricles. The QRS complex may include three waves: Q wave, R wave, and S wave. The T-wave may represent ventricular repolarization (recovery) as the ventricles prepare for the next contraction. The U-wave may sometimes be present after the T wave, it represents repolarization of the Purkinje fibers. The intervals between these waves may provide information about the duration and regularity of various phases of the cardiac cycle. Without limitation, the electrocardiogram signals 622 may be collected from the body using surface electrodes. In a non-limiting example, the surface electrodes may include small adhesive patches placed on the skin at specific locations on the chest, arms, and legs. Continuing, these electrodes may act as sensors that detect the electrical signals generated by the heart's activity as it beats.

With continued reference to FIG. 6, the plurality of voltage profiles 620 may include a frequency 624 corresponding to the at least a surface potential signal 610 associated with a temporal datum 626. As used in this disclosure, a “frequency” is the number of occurrences of a repeating event per unit of time. Without limitation, the frequency 624, may include how often a waveform or a voltage profile cycles within a given time frame, typically measured in Hertz (Hz), where one Hertz equals one cycle per second. In a non-limiting example, the frequency 624 may correspond to the cycles or oscillations of the at least a surface potential signal 610 over time, captured in relation to a temporal datum 626. For instance, if a potential signal oscillates 600 times per second, the frequency 624 of the signal is 600 Hz. Continuing, the temporal datum 626 in this case provides the exact timing reference for when those oscillations occurred, helping to track the signal's behavior over time for precise analysis. In another non-limiting example, frequency 624 may include the heart rate signal, where the oscillation of electrical pulses corresponds to each heartbeat, and the temporal datum 626 allows for correlating each pulse with the exact time it was recorded. As used in this disclosure, a “temporal datum” is a specific point in time associated with a particular event, signal, or measurement. In a non-limiting example, the temporal datum 626 may provide a reference to the exact timing of a signal. Without limitation, the temporal datum 626 may help in tracking when the signal was captured, changed, or measured. Continuing, the temporal datum 626 may allow for precise analysis and correlation of signals with time-based events or phenomena.

Still referring to FIG. 6, processor 602 is configured to select an optimal lead 628 comprising the at least a surface electrode pair 606. As used in this disclosure, an “optimal lead” is a specific electrode configuration or position that provides the most accurate or desirable signal quality. In a non-limiting example, the optimal lead 628 may provide benefits when recording or analyzing electrical activity. Without limitation, the optimal lead 628 may be chosen based on its ability to capture clear, reliable data that best represents the underlying physiological process, such as cardiac rhythms or electrical conduction. In a non-limiting example, the processor 602 may select a small number of optimal ECG or EGM leads to reduce the total number of leads required for accurate template matching. Continuing, the selection of the small number of optimal ECG or EGM leads may aid in streamlining the data collection process while still ensuring that critical information is captured effectively. Without limitation, the processor 602 may select the optimal leads 628 by choosing leads that demonstrate the highest signal magnitude. Continuing, the leads with stronger signals may provide clearer and more reliable data for analysis, which may be essential for accurate template matching. Continuing, the approach of selecting the optimal leads 628 may ensure that the most meaningful electrical activity is prioritized in the lead selection. Additionally, and or alternatively, selecting the optimal leads 628 may include selecting leads that, on average, show the least correlation with other selected optimal leads 628. Without limitation, by minimizing redundancy in the signals, the overall efficiency of the process is improved, reducing unnecessary data overlap. Continuing, the combination of high-signal leads with low correlation optimizes computational efficiency while maintaining the accuracy and reliability of the template matching process as further discussed herein. As described with reference to FIGS. 7A-7C, an eigenvector method may be utilized to determine the optimal lead 628.

With continued reference to FIG. 6, the optimal lead 628 may correspond to a maximum amplitude 630 in the at least a surface potential signal 610. Without limitation, the optimal lead 628 may include the lead that is positioned or oriented in such a way that it captures the maximum amplitude 630 in the at least a surface potential signal 610. Continuing, the optimal lead 628 may include the lead is configured to detect the strongest electrical signal possible in the given setup, ensuring that the most prominent data from the system (e.g., heart, muscle, or other structures) is recorded. Continuing, by selecting the optimal lead 628 the system 600 may enhance the accuracy of diagnostics and/or analysis. Without limitation, the maximum amplitude 630 may be calculated at a point in the signal, corresponding to a specific time or feature of interest. In another non-limiting example, the maximum amplitude 630 may be calculated over a region and subsequently averaged to ensure robustness against noise or localized signal variations.

With continued reference to FIG. 6, a downstream device 632 may be configured to display the optimal lead 628. As used in this disclosure, “downstream device” is a device that accesses and interacts with system 600. For instance, and without limitation, downstream device 632 may include a remote device and/or system 600. In a non-limiting embodiment, downstream device 632 may be consistent with a computing device as described in the entirety of this disclosure. Without limitation, the downstream device 632 may include a display device. As used in this disclosure, a “display device” refers to an electronic device that visually presents information to the entity. In some cases, display device may be configured to project or show visual content generated by computers, video devices, or other electronic mechanisms. In some cases, display device may include a liquid crystal display (LCD), a cathode ray tube (CRT), a plasma display, a light emitting diode (LED) display, and any combinations thereof. In a non-limiting example, one or more display devices may vary in size, resolution, technology, and functionality. Display device may be able to show any data elements and/or visual elements as listed above in various formats such as, textural, graphical, video among others, in either monochrome or color. Display device may include, but is not limited to, a smartphone, tablet, laptop, monitor, tablet, and the like. Display device may include a separate device that includes a transparent screen configured to display computer generated images and/or information. In some cases, display device may be configured to present a graphical user-interface (GUI) to a user, wherein a user may interact with a GUI. In some cases, a user may view a GUI through display. Additionally, or alternatively, processor 602 be connected to display device. In one or more embodiments, transmitting the optimal lead 628 may include displaying the optimal lead 628 at display device using a visual interface. A “graphical user interface,” as used herein, is a graphical form of user interface that allows users to interact with electronic devices. In some embodiments, GUI may include icons, menus, other visual indicators or representations (graphics), audio indicators such as primary notation, and display information and related user controls. A menu may contain a list of choices and may allow users to select one from them. A menu bar may be displayed horizontally across the screen such as pull-down menu. When any option is clicked in this menu, then the pull-down menu may appear. A menu may include a context menu that appears only when the user performs a specific action. An example of this is pressing the right mouse button. When this is done, a menu may appear under the cursor. Files, programs, web pages and the like may be represented using a small picture in a graphical user interface. For example, links to decentralized platforms as described in this disclosure may be incorporated using icons. Using an icon may be a fast way to open documents, run programs etc. because clicking on them yields instant access. Without limitation, the downstream device 632 may include various types of devices capable of presenting the data visually, such as a monitor, a graphical user interface (GUI), a touchscreen, or specialized diagnostic equipment integrated into medical or analytical systems. The device 632 may provide a clear and detailed visualization of the optimal lead 628, highlighting its selection as the lead that demonstrates the maximum signal amplitude or other selection criteria, such as minimal correlation with other leads or enhanced signal clarity. In some embodiments, the downstream device 632 may present the optimal lead 628 alongside additional contextual information, such as time-series data, signal strength comparisons, or overlays of other potential leads. This comprehensive view may enable users to better understand why a particular lead was selected as optimal. Furthermore, the displayed information may include graphical representations, such as waveforms, amplitude plots, or correlation matrices, which allow users to analyze the characteristics of the optimal lead 628. Continuing, the downstream device 632 may also support interactive functionality, allowing users to adjust parameters, filter the displayed data, or perform additional analyses. For instance, users might interact with the system to explore how different signal features or thresholds influence the selection of the optimal lead 628. Additionally, and/or alternatively, the device 632 may integrate diagnostic tools, such as annotations or alerts, to draw attention to critical features of the displayed data that may require further investigation.

Still referring to FIG. 6, processor 602 is configured to identify a template beat 634, using the optimal lead 628, wherein the template beat 634 comprises a first signal window 636 of a plurality of signal windows 638 of the first voltage profile 618. Without limitation, identifying the template beat 634 may include analyzing the electrical signal corresponding to the heart's activity to pinpoint the precise start and end of a specific cardiac event, such as a P wave or a QRS Complex. Continuing, once the cardiac event is identified, the waveform may be isolated, or windowed. Without limitation, “windowed” is when a specific portion 640 of a signal is extracted. Without limitation, the P wave or a QRS Complex may be windowed to create a clear representation of the P wave or QRS for use as a reference, or a template beat 634. As used in this disclosure, a “signal window” is a defined segment of a signal, selected over a specific time interval, that captures a portion 640 of the signal. Without limitation, the signal window may include an isolated and/or particular cardiac event, such as a P wave or QRS Complex from an EKG, for further study. Continuing, the signal window may be a windowed portion 640 of the signal used to focus on the important features while excluding surrounding data that may not be relevant to the analysis, aiding in tasks such as the creation of the template beat 634, signal comparison, and anomaly detection in signal data. As used in this disclosure, a “template beat” is a predefined, representative example of a specific cardiac event. In a non-limiting example, the template beat 634 may include the voltage profile of a cardiac event such as a P wave or QRS Complex, derived from one or more EKG leads. In a non-limiting example, the template beat 634 may be created by capturing the characteristic waveform, or windowing, of a cardiac event such as the P wave and/or the QRS Complex, which may serve as a reference, benchmark, and/or comparison point. Continuing, subsequent cardiac beats may be compared against the template beat 634 to assess consistency, detect abnormalities, and/or identify specific patterns in the heart's electrical activity. In a non-limiting example, the template beat 634 may be derived from the analysis of electrical signals detected by the system, such as ECG and/or EGM signal data.

With continued reference to FIG. 6, as a nonlimiting example, the template beat described herein may be consistent with the template beat disclosed in U.S. patent application Ser. No. 19/048,635 (attorney docket number 1518-172USU1), filed on Feb. 7, 2025, entitled “APPARATUS AND METHOD FOR BEAT MATCHING DURING ELECTROANATOMICAL MAPPING”, the entirety of which is incorporated herein by reference.

With continued reference to FIG. 6, a template beat 634 may include a QRS Complex. Without limitation, the template beat 634 may include the “template P,” the “template QRS,” and the like. Continuing, the “template P” and/or the “template QRS” may serve as benchmarks during cardiac monitoring and/or diagnostic procedures. In a non-limiting example, a template QRS Complex may be established based on a patient's baseline rhythm at the start of an electrophysiological study. Continuing, template QRS Complex may be used to identify any deviations during arrhythmia mapping and/or ablation procedures. For instance, a premature ventricular contraction (PVC) could be compared against the template QRS Complex to highlight differences in timing, amplitude, or morphology. In another non-limiting example, a “template T wave” may be used to monitor repolarization patterns in patients undergoing stress testing. Continuing, each detected T wave may be compared to the template T wave to detect potential ischemic changes or other abnormalities. Without limitation, the template beat 634 may provide a structured method for systematically comparing and detecting subtle variations in the electrical activity of the heart across multiple beats and leads.

With continued reference to FIG. 6, without limitation template beat 634 may include single lead templates and multi lead templates. In a non-limiting example, a single lead template may be derived from the electrical signals detected by a single lead of an electrocardiogram. Continuing, the single lead template may capture the characteristic features of a typical heartbeat, such as the shape, duration, and amplitude of the P wave, QRS Complex, and T wave, as observed from that specific lead. Without limitation, the single lead template may be used by the beat detection algorithm to identify and compare individual heartbeats detected by the same lead during electroanatomic mapping. Continuing, this approach ensures that the detected beats are accurately represented in both the geometric and electrical aspects of the map, providing a reliable reference for identifying deviations or abnormalities in the heart's electrical activity. In another non-limiting example, the multi-lead template may be created by graphing the electrical signals from multiple leads of an ECG. Continuing, the multi-lead template may capture the common features of a typical heartbeat as observed from various perspectives around the heart, providing a more comprehensive representation of the cardiac cycle. Without limitation, the multi-lead template may be used by the beat detection algorithm to identify and compare individual heartbeats detected by multiple leads during electroanatomic mapping. Without limitation, the multi-lead template may enhance the accuracy and reliability of beat detection, allowing for a more detailed analysis of the heart's electrical activity.

With continued reference to FIG. 6, the processor 602 may analyze the first voltage profile 618, which may represent the electrical activity of the heart, to detect a specific cardiac event, such as a P wave or QRS Complex. Continuing, the processor 602 may then isolate and capture the event within a defined time segment, referred to as the first signal window 636. Continuing, the signal window may encompass the portion 640 of the voltage profile that represents the desired characteristics of the cardiac event, such as its amplitude, shape, and duration. Without limitation, the identified template beat 634 may serve as a reference for comparing and analyzing subsequent cardiac events during the procedure.

With continued reference to FIG. 6, a signal window of the plurality of signal windows 638 may include a portion 640 of a voltage profile of the plurality of voltage associated with the temporal datum 626. As used in this disclosure, a “portion” is a specific part or segment of a larger whole. In a non-limiting example, the portion 640 may include a section of a voltage profile that is included within a signal window from the plurality of signal windows 638. Continuing, the portion 640 of the voltage profile may be associated with a particular temporal datum 626, meaning it may capture the electrical signal's characteristics (such as amplitude or frequency) during a specific time frame. Without limitation, the portion 640 of the voltage profile of the plurality of voltage profiles 620 may allow for a focused analysis of the signal during that period. In a non-limiting example, an electrical signal may be measured from a cardiac electrode during a heartbeat. Continuing, the signal window may isolate the time frame of 0.5 to 1.0 seconds, where the heart's electrical activity is at its peak. Continuing, within the signal window, a portion 640 of the voltage profile represents the electrical activity of the heart's ventricles during depolarization. Continuing, the temporal datum 626 may provide the exact time reference for this signal, allowing for precise analysis of the voltage behavior at that specific moment in the heart's cycle.

Still referring to FIG. 6, processor 602 is configured to automatically align, using the template beat 634 and a beat matching algorithm 642, a second signal window 644 of a second voltage profile 646 of the at least a surface potential signal 610 to the first signal window 636 of the first voltage profile 618 to determine a first matched beat 648. Without limitation, the processor 602 may utilize the previously identified template beat 634 from the first voltage profile 618 as a reference or benchmark. Continuing, the processor 602 may then apply the beat matching algorithm 642 to compare the second signal window 644, which represents a new segment of the second voltage profile 646, to the first signal window 636. Continuing, the beat matching algorithm 642 may ensure that the second signal window 644 is properly aligned with the template beat 634 by comparing features such as shape, timing, and/or amplitude. Continuing, once aligned, the processor 602 may determine whether the second signal window 644 sufficiently matches the template beat 634, resulting in the identification of a first matched beat 648. Continuing, this process may be used to assess the similarity between cardiac events and to detect patterns or abnormalities during a given cardiac procedure. As used in this disclosure, a “beat matching algorithm” is a computational method used to compare a detected cardiac beat to a predefined template beat 634. In a non-limiting example, the beat matching algorithm 642 may align the detected cardiac beat and the template beat 634 based on their features such as timing, shape, and/or amplitude. Without limitations, the beat matching algorithm 642 may evaluate the similarity between the two beats and may adjust for any variations in their positions or characteristics, in order to determine how closely the detected beat matches the template beat 634. In a non-limiting example, the beat matching algorithm 642 may process subsequent beats by analyzing each new beat within a signal window. Continuing, the beat matching algorithm 642 may compare specific features of the new beat to those of the template beat 634, such as the timing of peaks (e.g., the R wave) and troughs, the overall duration of the waveform, the height or amplitude of key points in the waveform, and the like. Continuing, the beat matching algorithm 642 may evaluate the absolute differences in these key features, aligning the beats based on their timing and feature set. For example, without limitation, the beat matching algorithm 642 may compare the time at which the R wave occurs in the new beat relative to the template, adjusting for any time shifts to ensure the beats are properly aligned. Continuing, the beat matching algorithm 642 may then assess whether the amplitude of the R wave and other features, like the shape of the QRS Complex, fall within an acceptable range of similarity as discussed in more detail below. Continuing, if the timing, shape, and amplitude of the new beat closely match those of the template beat 634 within predefined thresholds, the beat matching algorithm 642 may designate it as a “matched beat.” Continuing, if significant deviations are found, such as abnormal timing or an unusually shaped waveform, the beat may be flagged for further analysis as a potential abnormality. Without limitation, the beat matching algorithm 642 may automatically match beats based on specific, comparable features, ensuring accurate detection and alignment of cardiac activity. As used in this disclosure, a “matched beat” is a detected cardiac beat that has been aligned and compared to a template beat 634 using a beat matching algorithm 642. In a non-limiting example, the matched beat may be determined to sufficiently correspond to the template beat 634. Without limitation, a matched beat may exhibit similar characteristics to the template beat 634, such as waveform morphology, timing, and signal amplitude, indicating that it belongs to the same type of cardiac event.

With continued reference to FIG. 6, as a nonlimiting example, the beat matching algorithm described herein may be consistent with the beat matching algorithm disclosed in U.S. patent application Ser. No. 19/048,635 (attorney docket number 1518-172USU1), filed on Feb. 7, 2025, entitled “APPARATUS AND METHOD FOR BEAT MATCHING DURING ELECTROANATOMICAL MAPPING”, the entirety of which is incorporated herein by reference.

With continued reference to FIG. 6, the system 600 may be further configured to generate a similarity signal 650 by comparing the first signal window 636 of the first voltage profile 618 and the second signal window 644 of the second voltage profile 646, provide, using at least a visual element 652, feedback 654 of a degree of match 656, and determine, using the at least a processor, a threshold 658 for detecting a plurality of matched beats 664 as a function of one or more statistical values 660 calculated based on a specific metric 662. As used in this disclosure, a “similarity signal” is a metric generated by comparing the first signal window 636 of the first voltage profile 618 with the second signal window 644 of the second voltage profile 646. In a non-limiting example, the similarity signal 650 may quantify how closely the two signals align over time by measuring the degree of similarity or dissimilarity between them. Continuing, the process may involve aligning the template beat 634 with each sample of the second signal window 644, one sample at a time, and calculating how well the two signals match at each time point.

With continued reference to FIG. 6, the system 600 may further include computing a sum of squared differences of the template beat 634 and the processed potential signal to generate the similarity signal 650. As used in this disclosure, a “sum of squared differences” (SSD) is a mathematical calculation that quantifies the difference between two signals by comparing their values at corresponding points. Without limitation, for each point in the signals being compared, the difference between the values is squared to eliminate negative differences and emphasize larger deviations. Without limitation, the squared differences for all points in the signal may then be summed to produce a single value. Continuing, the single value may represent the overall dissimilarity between the two signals. Without limitation, a lower sum may indicate that the signals are closely matched. Without limitation, a higher sum may indicate a greater difference in the signals and that they are not closely matched. In a non-limiting example, the sum of squared differences may be used in signal processing to assess how well two signals align, such as comparing a template ECG beat to a new beat being analyzed.

With continued reference to FIG. 6, in a non-limiting example, the process of generating the similarity signal 650 may include identifying a template beat 634 is a QRS Complex with a peak amplitude of 1 mV and a duration of 600 ms. Continuing the template beat 634 may be extracted from a reference ECG recording during normal sinus rhythm and may contain the characteristic sharp upward spike of the R wave. Continuing, the second signal window 644 may include a portion 640 of an ECG recording being analyzed, where the beat matching algorithm 642 may align the template beat 634 with each beat in the signal window, one sample at a time, across the entire length of the recording. For instance, the second signal window 644 may include 500 ms of recorded ECG data that includes normal beats and a potential premature ventricular contraction (PVC). Continuing, as the beat matching algorithm 642 shifts the template beat 634 (the template QRS Complex) across the signal window, the beat matching algorithm 642 may calculate the sum of squared differences at each time point. Continuing, consider the template QRS aligned with a normal beat at time point 200 ms. The sum of squared differences between the template QRS and the signal at this point might be very low, resulting in a value close to zero, indicating a strong match. Continuing, this may produce a sharp downward deflection in the similarity signal 650. Without limitation, when the beat matching algorithm 642 aligns the template QRS Complex with a PVC occurring at time point 350 ms, the QRS morphology in the signal window may differ from the template (e.g., wider QRS, lower amplitude). Continuing, as a result, the sum of squared differences may increase thereby producing a higher value in the similarity signal 650. Continuing, this may be reflected in the detection signal as a smaller deflection or even an upward spike, indicating a weaker match. Continuing, across the entire ECG recording, the similarity signal 650 may show multiple downward deflections, each corresponding to a match between the template and the recorded beats. Without limitation, the sharpest and lowest deflection may occur at time points where the match is closest (for normal beats), while higher values will appear for mismatches (such as the PVC). For example, without limitation, if a perfect match occurs at 200 ms, the similarity signal 650 may hit zero, but when aligned with the PVC at 350 ms, the value may be 0.5, indicating a less precise match.

With continued referent to FIG. 6, as used in this disclosure, “feedback” is information provided to the user that indicates the result of the analysis of the degree of match 656 between the template beat 634 and detected beats. Continuing, feedback 654 may be displayed visually through graphical elements or indicators, such as waveform displays, numerical values, or color-coded signals, and it may allow the user to assess how closely the beats align. Without limitation, feedback 654 may help guide decisions during the procedure by showing whether the detected beats are sufficiently similar to the template or if further adjustments are needed. As used in this disclosure, a “degree of match” is a quantifiable measurement that indicates how closely a detected beat aligns with a template beat. Without limitation, the degree of match 656 may be determined by the beat matching algorithm and may be represented by a numerical value or visual cue. Continuing, a lower value or closer visual alignment suggests a stronger match, while a higher value or misalignment indicates a weaker or less accurate match.

Still referring to FIG. 6, processor 602 is configured to determine, using the at least a processor 602, a threshold 658 for detecting a plurality of matched beats 664 as a function of one or more statistical values 660 calculated based on a specific metric 662. As used in this disclosure, a “threshold” is a predetermined value that serves as a cutoff point for detecting a plurality of matched beats 664. In a non-limiting example, the threshold 658 may be set to differentiate between signals that match the template beat 634 and those that do not. Continuing, the threshold 658 may act as a cutoff point, so that only signals with a degree of match 656 that falls below the predefined value are considered sufficiently similar to the template beat 634. Continuing, threshold 658 may help filter out less relevant or dissimilar beats, allowing the system 600 to focus on the target beats that meet the criteria of similarity. As used in this disclosure, “statistical values” are numerical measures derived from analyzing data over several beats. In a non-limiting example, the statistical values 660 may be used to inform decision-making processes. In a non-limiting example, the statistical values 660 may include measures such as the median, mean, standard deviation, or other statistical descriptors that represent the behavior of the detection signal over time. In a non-limiting example, the statistical values 660 may be calculated based on the observed metrics from a group of beats and are used to set the threshold 658 for beat detection. As used in this disclosure, a “specific metric” is a quantifiable feature or characteristic of a signal that is used to assess the degree of match 656 between a template beat 634 and a detected beat. In a non-limiting example, the specific metric 662 may include the sum of squared differences or another measurement of how closely the signals align. For example, without limitation, the median value of the detection signal, or a similar baseline measure, may be used as the specific metric 662 to determine how closely the beats match the template beat 634. Continuing, beats that fall below a certain percentage of this median value may be considered matches, allowing flexibility in how strictly the system identifies beats, based on the case or procedure requirements. Continuing, this process may allow the physician to adjust the threshold 658 for more precise or broader beat matching, depending on the needs of the procedure.

With continued reference to FIG. 6, the threshold 658 may be calculated based on a median value of the similarity signal. As used in this disclosure, a “median value” is the middle value in a set of numerical data when the values are arranged in ascending or descending order. Continuing, if the data set has an odd number of values, the median is the value that falls exactly in the middle. Continuing, if the data set has an even number of values, the median is the average of the two middle values. Without limitation, the median value may be useful for determining a central tendency that is less affected by extreme outliers compared to the mean, making it a robust measure for defining threshold 658 in signal analysis. In a non-limiting example, with continued reference to FIG. 6, the threshold 658 may be calculated using the median value of the similarity signal. For instance, without limitation, if the similarity signal, generated by comparing multiple beats to the template beat 634, ranges from 0 to 1 (where 0 represents a perfect match), the system 600 may collect data from 50 consecutive beats. Continuing, the similarity values for these beats may range from 0.1 to 0.7, with a median value of 0.4. Continuing, based on this median value, the threshold 658 for detecting target beats may be set at 80% of the median value, meaning that only beats with a similarity signal below 0.32 (0.8×0.4) would be classified as matched beats. Continuing, this would allow the system to filter out beats that are less similar to the template, ensuring that only the closest matches are identified. Additionally and or alternatively, if the physician desires a more lenient matching, the threshold 658 may be set at 620% of the median value, selecting beats with a similarity signal below 0.48.

With continued reference to FIG. 6, the beat matching algorithm 642 may detect a plurality of matched beats 664 by overlapping the template beat 634 of the first voltage profile 618 over a second voltage profile 646 and aligning the template beat 634 with the second signal window 644 of the second voltage profile 646 as a function of the similarity signal 650. Without limitation, the template beat 634, which may have been previously identified from the first voltage profile 618, may be continuously shifted and overlapped with different sections of the second voltage profile 646. Continuing, for each position, the beat matching algorithm 642 may calculate the similarity signal 650, which reflects how well the template beat 634 aligns with the new section, or the second signal window 644, of the second voltage profile 646. Continuing, as the beat matching algorithm 642 shifts the template across the second signal window 644, it may identify points where the similarity signal 650 indicates a close match between the two signals. For each detected match, the beat matching algorithm 642 may flag a matched beat, and this process is repeated across the entire second voltage profile 646, resulting in the detection of a plurality of matched beats 664. Without limitation, this technique may ensure that the beat matching algorithm 642 can automatically identify instances where the template beat 634 is present within the second voltage profile 646, allowing for efficient detection and comparison of cardiac events.

With continued reference to FIG. 6, the beat matching algorithm 642 may detect a plurality of matched beats 664 by overlapping the template beat 634 of the first voltage profile 618 over the second voltage profile 646 and aligning the template beat 634 with the second signal window 644 of the second voltage profile 646 as a function of the similarity signal 650. Without limitation, the template beat 634, which may have been previously identified from the first voltage profile 618, may be continuously shifted and overlapped with different sections of the second voltage profile 646. Continuing, for each position, the beat matching algorithm 642 may calculate the similarity signal 650, which reflects how well the template beat 634 aligns with the new section, or the second signal window 644, of the second voltage profile 646. Continuing, as the beat matching algorithm 642 shifts the template across the second signal window 644, it may identify points where the similarity signal 650 indicates a close match between the two signals. For each detected match, the beat matching algorithm 642 may flag a matched signal, and this process is repeated across the entire second voltage profile 646, resulting in the detection of a plurality of matched signals. Without limitation, this technique may ensure that the beat matching algorithm 642 can automatically identify instances where the template beat 634 is present within the second voltage profile 646, allowing for efficient detection and comparison of cardiac events.

Referring now to FIG. 7A, an exemplary illustration 700a of a first eigenvector distribution comprising optimal orthogonal leads, two lateral midlines, and an anterior midline. As used in this disclosure, an “eigenvector distribution” is the arrangement or spread of the eigenvectors associated with a matrix. In an embodiment, the first eigenvector distribution 704a may reflect the primary directional components derived from a set of transformations or data specific to the system, such as a human torso. As used in this disclosure, “lateral midline” is the imaginary line or axes that run along the lateral (side) aspect of a structure or object. In an embodiment, the lateral midlines 208 may divide a structure into symmetrical or functionally relevant parts. In an embodiment, the lateral midlines 208 may include the lines running along the sides of a human body. Continuing, the lateral midlines 208 may help define anatomical positions or guide surgical incisions. In an embodiment, illustration 700a may include two lateral midlines 208 representing the sides of a human torso region.

With continued reference to FIG. 7A, in an embodiment, the anterior midline 712 may divide a structure or object into two symmetrical halves. In an embodiment, the anterior midline 712 may divide the human body from the head down to the feet, passing through the middle of structures such as the nose, sternum, and navel. In an embodiment, the anterior midline 712 may serve as a reference point for describing locations, movements, or conducting medical procedures along the front surface of the body. In an embodiment, the illustration 700a depicts one anterior midline 712 in the middle of the eigenvector distribution 704a that represents the centerline down the middle of a human torso.

With continued reference to FIG. 7A, as used in this disclosure, an “optimal orthogonal lead” is a lead vector or axis that is both orthogonal to each other and chosen in such a way that they maximize a specific desired outcome or performance criterion. In an embodiment, the eigenvector distribution 704a may include two optimal orthogonal leads 716. In an embodiment, the optimal orthogonal leads 716 may capture or measure signals from distinct, non-overlapping dimensions with the highest efficiency or accuracy. For example, without limitation, the optimal orthogonal lead may be configured to provide the best possible view of electrical activity in the heart, ensuring that the data is captured from independent planes without redundancy. In an embodiment, the illustration 700a includes a human body 728 to depict the location of the eigenvector distribution 704a. In an embodiment, the illustration 700a includes a transversal axis 732 that runs horizontally across the human body 728. In an embodiment, the illustration 700a includes a posterior midline 736. In an embodiment, the identification of the optimal orthogonal lead 716 may be a critical step in determining the optimal lead, as previously described with reference to FIG. 6.

Referring now to FIG. 7B, an exemplary illustration 700b of a second eigenvector distribution comprising optimal orthogonal leads, vectorcardiography leads, and 12-lead electrocardiogram leads. In an embodiment, the second eigenvector distribution 704b may be the arrangement or orientation of the second most significant eigenvector derived from a matrix or system. In an embodiment, the second eigenvector distribution 704b may capture the next most important direction of influence after the first eigenvector distribution 704a, providing additional insights into the behavior or characteristics of the system. In an embodiment, the second eigenvector distribution 704b may include two optimal orthogonal leads 716, 12-lead electrocardiogram leads 720, and four vectorcardiography leads 724.

With continued reference to FIG. 7B, as used in this disclosure, “12-lead electrocardiogram leads” are a specific configuration of electrodes placed on the body to capture electrical signals generated by the heart from multiple angles. In an embodiment, the 12-lead electrocardiogram leads 720 provide a comprehensive view of the heart's electrical activity by recording data from 12 different perspectives, allowing for detailed analysis of the heart's rhythm, rate, and electrical conduction pathways.

With continued reference to FIG. 7B, as used in this disclosure, a “vectorcardiography lead” is an electrode used in vectorcardiography. Vectorcardiography is a method for recording the electrical activity of the heart by measuring the magnitude and direction of the heart's electrical forces as vectors. Continuing, unlike an electrocardiogram, which records the heart's electrical activity in a time-based graph, a vectorcardiography creates a three-dimensional representation of the heart's electrical conduction. In an embodiment, the illustration 700b includes a human body 728 to depict the location of the eigenvector distribution 704b. In an embodiment, the illustration 700b includes a transversal axis 732 that runs horizontally across the human body 728. In an embodiment, the illustration 700a includes a posterior midline 736.

Referring now to FIG. 7C, an exemplary illustration 700c of a second eigenvector distribution comprising optimal orthogonal leads, vectorcardiography leads, and 12-lead electrocardiogram leads. In an embodiment, the third eigenvector distribution 704c is the arrangement or orientation of the third most significant eigenvector derived from a matrix or system. Continuing, the third eigenvector distribution 704c may capture the next most important direction of variation or influence after the first eigenvector distribution and the second eigenvector distribution. In a non-limiting example, the first eigenvector distribution and the second eigenvector distribution may provide primary insights into the dominant behaviors or patterns in the system and the third eigenvector distribution 704c may reveal additional, often subtler, dimensions of variation. In an embodiment, the third eigenvector distribution 704c may include two optimal orthogonal leads 716 as previously described herein. In an embodiment, the illustration 700c includes a human body 728 to depict the location of the eigenvector distribution 704c. In an embodiment, the illustration 700c includes a transversal axis 732 that runs horizontally across the human body 728. In an embodiment, the illustration 700a includes a posterior midline 736.

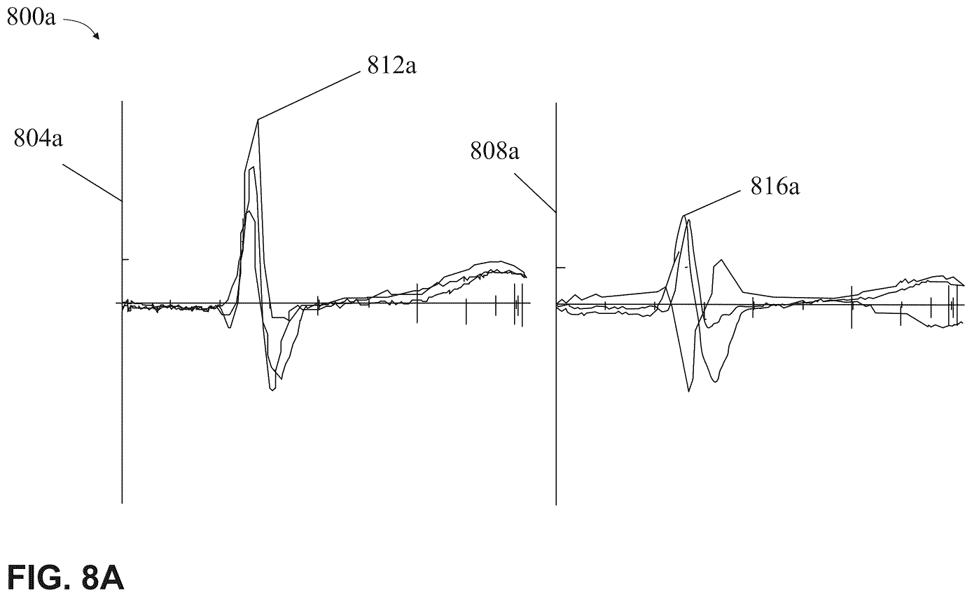

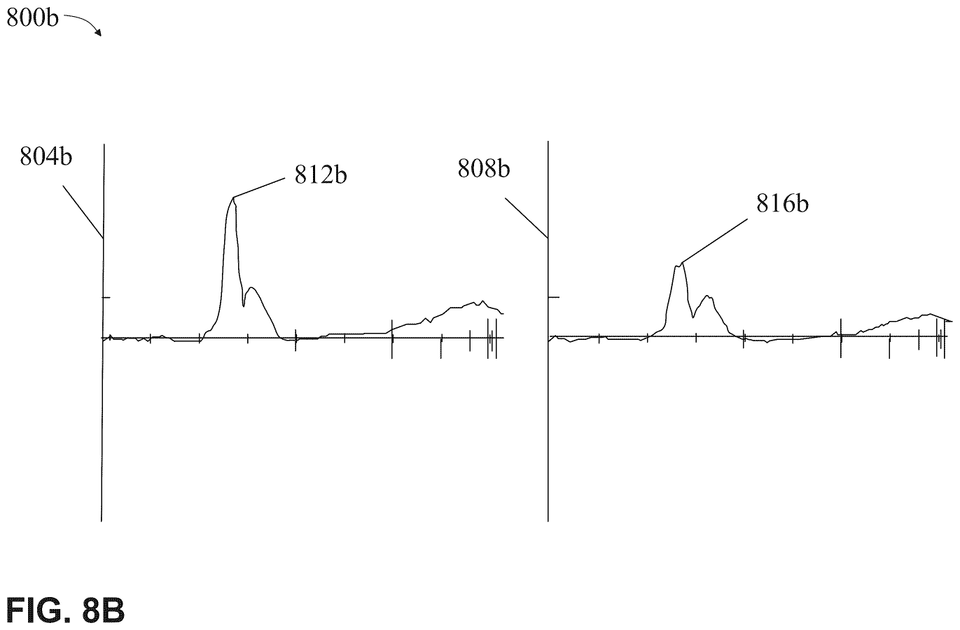

Referring now to FIG. 8A, an exemplary illustration 300a of a comparison of lead graphs of optimal leads and vectorcardiography leads. In an embodiment, the illustration 300a includes a graph of optimal leads 304a. In an embodiment, the illustration 300a includes a graph of vectorcardiography leads 308a. In an embodiment, the graph of optimal leads 304a includes an optimal lead peak point 312a. In an embodiment, the graph of vectorcardiography leads 308a includes a vectorcardiography lead peak point 316a.

Referring now to FIG. 8B, an exemplary illustration 800b of a comparison of graphs of root mean square (RMS) values of optimal leads and vectorcardiography leads. In an embodiment, the illustration 800b includes a graph of a root mean square value of optimal leads 804b. In an embodiment, the illustration 800b includes a graph of a root mean square value of vectorcardiography leads 808b. In an embodiment, the graph of the root mean square value of optimal leads 804b includes an optimal lead peak root mean square value 812b of 848 μV. In an embodiment, the optimal lead peak root mean square value 812b represents the maximum recorded RMS voltage for those leads. In an embodiment, the graph of the root mean square value of the vectorcardiography leads 808b includes a vectorcardiography lead peak root mean square value 816b of 179 μV. In an embodiment, the vectorcardiography lead peak root mean square value 316b represents the maximum RMS voltage recorded for those leads. In an embodiment, the optimal lead peak root mean square value 812b is 94% higher than the vectorcardiography lead peak root mean square value 816b.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

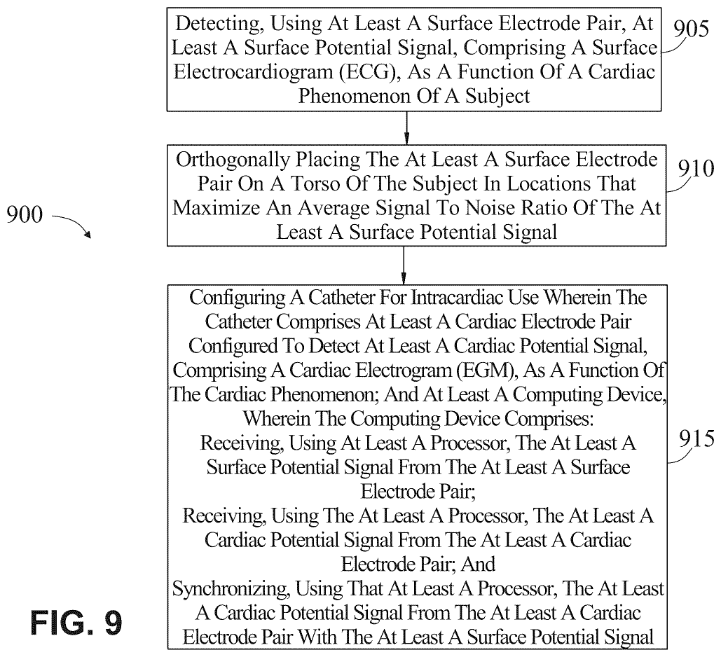

Referring now to FIG. 9, a flow diagram of an exemplary method 900 for determining a beat match using an optimal lead is illustrated. At step 905, method 900 includes receiving, using at least a processor, a first potential signal from at least an electrode, wherein the first potential signal comprise a first voltage profile of a plurality of voltage profiles. This may be implemented as described and with reference to FIGS. 1-8B.

Still referring to FIG. 9, at step 910, method 900 includes selecting, using the at least a processor, an optimal lead comprising the at least an electrode. This may be implemented as described and with reference to FIGS. 1-8B.

Still referring to FIG. 9, at step 915, method 900 includes identifying a template beat, using the optimal lead, wherein the template beat comprises a first signal window of a plurality of signal windows of the first voltage profile. This may be implemented as described and with reference to FIGS. 1-8B.

Still referring to FIG. 9, at step 920, method 900 includes automatically aligning, using the template beat and a beat matching algorithm, a second signal window of a second voltage profile of the potential signal to the first signal window of the first voltage profile to determine a first matched beat. This may be implemented as described and with reference to FIGS. 1-8B.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

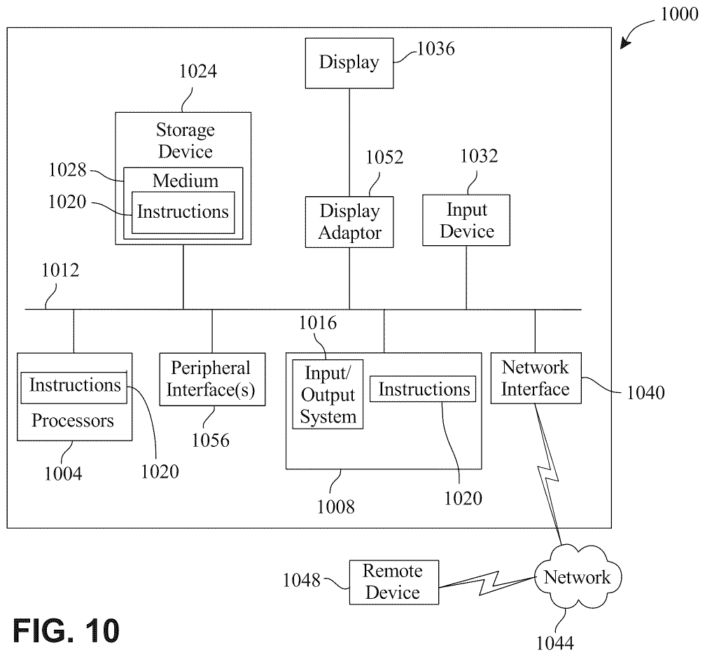

FIG. 10 shows a diagrammatic representation of one embodiment of computing device in the exemplary form of a computer system 1000 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1000 includes a processor 1004 and a memory 1008 that communicate with each other, and with other components, via a bus 1012. Bus 1012 may include any of several types of bus structures including, but not limited to, a memory bus, a memory controller, a peripheral bus, a local bus, and any combinations thereof, using any of a variety of bus architectures.

Processor 1004 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 1004 may be organized according to Von Neumann and/or Harvard architecture as a non-limiting example. Processor 1004 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), system on module (SOM), and/or system on a chip (SoC).

Memory 1008 may include various components (e.g., machine-readable media) including, but not limited to, a random-access memory component, a read only component, and any combinations thereof. In one example, a basic input/output system 1016 (BIOS), including basic routines that help to transfer information between elements within computer system 1000, such as during start-up, may be stored in memory 1008. Memory 1008 may also include (e.g., stored on one or more machine-readable media) instructions (e.g., software) 1020 embodying any one or more of the aspects and/or methodologies of the present disclosure. In another example, memory 1008 may further include any number of program modules including, but not limited to, an operating system, one or more application programs, other program modules, program data, and any combinations thereof.

Computer system 1000 may also include a storage device 1024. Examples of a storage device (e.g., storage device 1024) include, but are not limited to, a hard disk drive, a magnetic disk drive, an optical disc drive in combination with an optical medium, a solid-state memory device, and any combinations thereof. Storage device 1024 may be connected to bus 1012 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1024 (or one or more components thereof) may be removably interfaced with computer system 1000 (e.g., via an external port connector (not shown)). Particularly, storage device 1024 and an associated machine-readable medium 1028 may provide nonvolatile and/or volatile storage of machine-readable instructions, data structures, program modules, and/or other data for computer system 1000. In one example, software 1020 may reside, completely or partially, within machine-readable medium 1028. In another example, software 1020 may reside, completely or partially, within processor 1004.

Computer system 1000 may also include an input device 1032. In one example, a user of computer system 1000 may enter commands and/or other information into computer system 1000 via input device 1032. Examples of an input device 1032 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1032 may be interfaced to bus 1012 via any of a variety of interfaces (not shown) including, but not limited to, a serial interface, a parallel interface, a game port, a USB interface, a FIREWIRE interface, a direct interface to bus 1012, and any combinations thereof. Input device 1032 may include a touch screen interface that may be a part of or separate from display device 1036, discussed further below. Input device 1032 may be utilized as a user selection device for selecting one or more graphical representations in a graphical interface as described above.

A user may also input commands and/or other information to computer system 1000 via storage device 1024 (e.g., a removable disk drive, a flash drive, etc.) and/or network interface device 1040. A network interface device, such as network interface device 1040, may be utilized for connecting computer system 1000 to one or more of a variety of networks, such as network 1044, and one or more remote devices 1048 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1044, may employ a wired and/or a wireless mode of communication. In general, any network topology may be used. Information (e.g., data, software 1020, etc.) may be communicated to and/or from computer system 1000 via network interface device 1040.

Computer system 1000 may further include a video display adapter 1052 for communicating a displayable image to a display device, such as display device 1036. Examples of a display device include, but are not limited to, a liquid crystal display (LCD), a cathode ray tube (CRT), a plasma display, a light emitting diode (LED) display, and any combinations thereof. Display adapter 1052 and display device 1036 may be utilized in combination with processor 1004 to provide graphical representations of aspects of the present disclosure. In addition to a display device, computer system 1000 may include one or more other peripheral output devices including, but not limited to, an audio speaker, a printer, and any combinations thereof. Such peripheral output devices may be connected to bus 1012 via a peripheral interface 1056. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. An electroanatomical mapping system using an optimal lead, wherein the electroanatomical mapping system comprises:
at least a surface electrode pair configured to:
detect at least a surface potential signal, comprising a surface electrocardiogram (ECG), as a function of a cardiac phenomenon of a subject, wherein:
the ECG comprises electrical activity representing a plurality of heartbeats of the subject; and
the electrical activity of each heartbeat comprises a QRS complex which is a component of an electrical wave propagating through a heart of the subject; and
be orthogonally placed on a torso of the subject in locations that maximize an average signal to noise ratio of the at least a surface potential signal; and
at least a computing device, wherein the computing device comprises:
a memory; and
at least a processor communicatively connected to the memory, wherein the memory contains instructions configuring the at least a processor to:
receive the at least a surface potential signal from the at least a surface electrode pair, wherein receiving the at least a surface potential signal comprises receiving the QRS complex of each heartbeat of the subject;
identify a template QRS specifically corresponding to the cardiac phenomenon of the subject from a plurality of template beats;
align the template QRS to the QRS complex of each heartbeat of the subject using a beat matching algorithm based on one or more QRS wave features including timing, shape and amplitude;
generate a detection signal based on degree of similarity between the QRS complex of each heartbeat and the template QRS;
define a threshold metric for accepting heartbeats based on the detection signal to identify matched heartbeats; and
select, during an electrophysiologic (EP) procedure, only those heartbeats of the subject satisfying the threshold metric to analyze the cardiac phenomenon of the subject while ignoring other heartbeats.

2. The system of claim 1, wherein the surface electrode pair is configured to be disposed on the torso located at a first intersection on an anterior midline and slightly above a transversal plane and at a second intersection substantially between the anterior midline and a right lateral midline and slightly below the transversal plane.

3. The system of claim 2, wherein the surface electrode pair is configured to be disposed on the torso located at a third intersection slightly right of the anterior midline and substantially on the transversal plane and at a fourth intersection on a posterior midline and on the transversal plane.

4. The system of claim 2, wherein the surface electrode pair is configured to be disposed on the torso located at a fifth intersection slightly left of the anterior midline and moderately below the transversal plane and a sixth intersection moderately right of the anterior midline and moderately above the transversal plane.

5. The system of claim 1, wherein the optimal lead corresponds to a maximum amplitude in the at least a surface potential signal.

6. The system of claim 1, wherein a downstream device is configured to display the optimal lead.

7. The system of claim 1, wherein a signal window of a plurality of signal windows comprises a portion of a voltage profile of the plurality of voltage associated with a temporal datum.

8. The system of claim 7, wherein the system is further configured to:
generate a similarity signal by comparing a first signal window of a first voltage profile and a second signal window of a second voltage profile;
provide, using at least a visual element, feedback of a degree of match; and
determine, using the at least a processor, a threshold for detecting a plurality of matched beats as a function of one or more statistical values calculated based on a specific metric.

9. The system of claim 8, wherein the beat matching algorithm detects a plurality of matched beats by:
overlapping a template beat of the first voltage profile over the second voltage profile; and
aligning the template beat with the second signal window of the second voltage profile as a function of a similarity signal.

10. (canceled)

11. A method for an electroanatomical mapping system using an optimal lead, wherein the method comprises:
detecting, using at least a surface electrode pair, at least a surface potential signal, comprising a surface electrocardiogram (ECG), as a function of a cardiac phenomenon of a subject, wherein:
the ECG comprises electrical activity representing a plurality of heartbeats of the subject; and
the electrical activity of each heartbeat comprises a QRS complex which is a component of an electrical wave propagating through a heart of the subject;
orthogonally placing the at least a surface electrode pair on a torso of the subject in locations that maximize an average signal to noise ratio of the at least a surface potential signal;
receiving, using at least a processor, the at least a surface potential signal from the at least a surface electrode pair, wherein receiving the at least a surface potential signal comprises receiving the QRS complex of each heartbeat of the subject;
identifying, using the at least a processor, a template QRS specifically corresponding to the cardiac phenomenon of the subject from a plurality of template beats;
aligning, using the at least a processor, the template QRS to the QRS complex of each heartbeat of the subject using a beat matching algorithm based on one or more QRS wave features including timing, shape and amplitude;
generating, using the at least a processor, a detection signal based on degree of similarity between the QRS complex of each heartbeat and the template QRS;
defining, using the at least a processor, a threshold metric for accepting heartbeats based on the detection signal to identify matched heartbeats; and
select, using the at least a processor, during an electrophysiologic (EP) procedure, only those heartbeats of the subject satisfying the threshold metric to analyze the cardiac phenomenon of the subject while ignoring other heartbeats.

12. The method of claim 11, wherein the surface electrode pair is disposed on the torso located at a first intersection on an anterior midline and slightly above a transversal plane and at a second intersection substantially between the anterior midline and a right lateral midline and slightly below the transversal plane.

13. The method of claim 12, wherein the surface electrode pair is disposed on the torso located at a third intersection slightly right of the anterior midline and substantially on the transversal plane and at a fourth intersection on a posterior midline and on the transversal plane.

14. The method of claim 12, wherein the surface electrode pair is disposed on the torso located at a fifth intersection slightly left of the anterior midline and moderately below the transversal plane and a sixth intersection moderately right of the anterior midline and moderately above the transversal plane.

15. The method of claim 11, wherein the optimal lead corresponds to a maximum amplitude in the at least a surface potential signal.

16. The method of claim 11, wherein a downstream device is configured to display the optimal lead.

17. The method of claim 11, wherein a signal window of a plurality of signal windows comprises a portion of a voltage profile of the plurality of voltage associated with a temporal datum.

18. The method of claim 17 further comprising:
generating a similarity signal by comparing a first signal window of a first voltage profile and a second signal window of a second voltage profile;
providing, using at least a visual element, feedback of a degree of match; and
determining, using the at least a processor, a threshold for detecting a plurality of matched beats as a function of one or more statistical values calculated based on a specific metric.

19. The method of claim 18, wherein the beat matching algorithm detects a plurality of matched beats by:
overlapping a template beat of the first voltage profile over the second voltage profile; and
aligning the template beat with the second signal window of the second voltage profile as a function of a similarity signal.

20. (canceled)
